(19) United States
(12) Patent Application Publication
(10) Pub. No.: US 2025/0114354 A1
(43) Pub. Date: Apr. 10, 2025

(54) COMBINATION TREATMENT REGIMENS - SMARCA2 DEGRADER WITH PD-1 INHIBITORS

(71) Applicant: PRELUDE THERAPEUTICS INCORPORATED, Wilmington, DE (US)

(72) Inventors: Koichi Ito, Philadelphia, PA (US); Michael Hulse, Doylestown, PA (US); Peggy Scherle, Wayne, PA (US); Norman Fultang, Philadelphia, PA (US); Krishna Vaddi, Chadds Ford, PA (US)

(21) Appl. No.: 18/906,352

(22) Filed: Oct. 4, 2024

Related U.S. Application Data
(60) Provisional application No. 63/587,767, filed on Oct. 4, 2023.
(60) Provisional application No. 63/594,108, filed on Oct. 30, 2023.
(60) Provisional application No. 63/657,189, filed on Jun. 7, 2024.

(51) Int. Cl. A61K 31/5025; A61K 39/395; A61P 35/00

(52) U.S. Cl. CPC A61K 31/5025; A61K 39/3955; A61P 35/00

(57) ABSTRACT

Disclosed are methods of treating cancer comprising administering to a subject a treatment regimen comprising a compound of Formula (I):

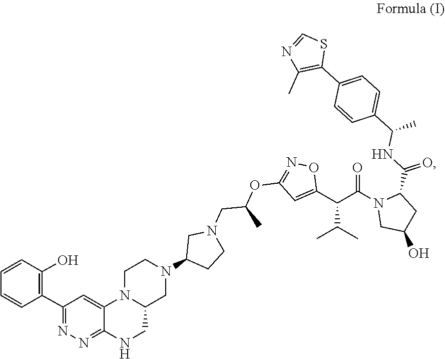 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

or a pharmaceutically acceptable salt thereof, and a PD-1 inhibitor.

### CROSS-REFERENCE TO RELATED APPLICATIONS

This application claims the benefit of U.S. Provisional Application No. 63/587,767, filed Oct. 4, 2023, and U.S. Provisional Application No. 63/594,108, filed Oct. 30, 2023, and U.S. Provisional Application No. 63/657,189, filed Jun. 7, 2024. Each of the aforementioned applications is incorporated by reference herein in its entirety.

### TECHNICAL FIELD

The disclosure pertains to methods of treating cancer comprising administering to a subject a treatment regimen comprising a SMARCA2 degrader and PD-1 inhibitor.

### BACKGROUND

SMARCA2 and SMARCA4 are the core catalytic subunits of the SWI/SNF complexes, which play an important role in controlling gene expression by remodeling chromatin. SMARCA4 is mutated in multiple cancers and SMARCA4-deficient cancer cells can become highly dependent on SMARCA2 for their survival. Therefore, targeting SMARCA2 in SMARCA4-deleted cancers using selective SMARCA2 degraders induces synthetic lethality, while sparing SMARCA4 wild-type normal cells. Cell lines with SMARCA4 damaging mutation or low expression show high SMARCA2 gene dependency scores, suggesting the synthetic lethal relationship of targeting SMARCA2 and SMARCA4-deficiency. SMARCA4 mutations occur in different types of cancer including lung adenocarcinoma, skin cancer/melanoma, uterine/endometrioid adenocarcinoma, bladder cancer, colon adenocarcinoma, among others. In addition to damaging mutation (“loss of SMARCA4 protein”), some tumors express SMARCA4 hotspot missense mutations near its ATP binding site or DNA binding site that likely alters biological function of SMARAC4.

The compound of Formula (I) is a SMARCA2 degrader:

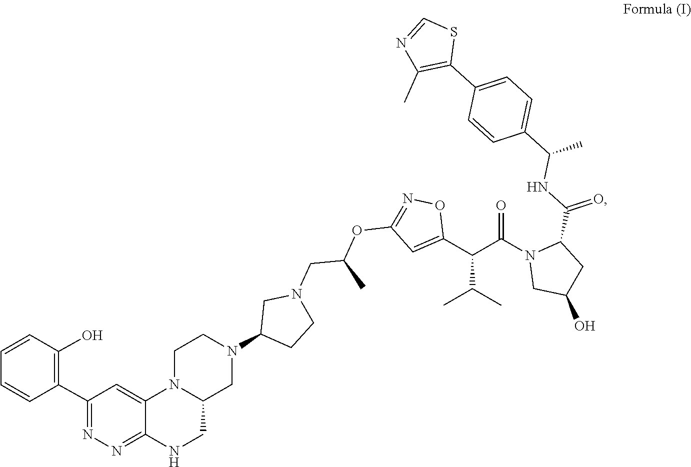 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

that was disclosed in WO2021252666. Formula (I) regulates cancer cell proliferation and growth through cell cycle arrest and DNA replication inhibition in SMARCA4 mutated cancer cells. Methods of making the compound of Formula (I) are known in the art, and include those set forth in WO2021252666.

Programmed cell death protein 1 (PD-1), a cell surface receptor on immune system T cells and B cells that has a role in regulating the immune system response by down-regulating the immune system and suppressing T cell inflammatory activity, thereby preventing autoimmune diseases. This same activity, however, can also prevent the immune system from killing cancer cells.

PD-1 inhibitors are drugs that that block PD-1, and thereby activate the immune system to attack tumor cells.

A need exists for treatment regimen comprising combination therapies for treating cancer that utilize both PD-1 inhibitors and other chemotherapeutic agents.

### SUMMARY

The disclosure provides methods of treating cancer in a subject in need thereof, comprising administering to the subject a treatment regimen comprising:
(a) a compound of Formula (I):

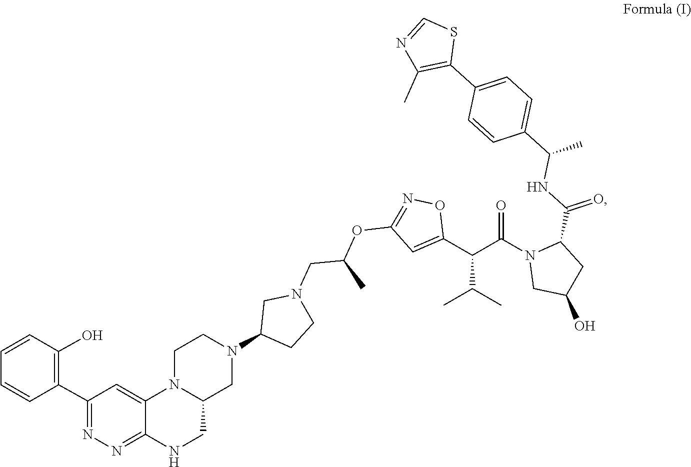 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

or a pharmaceutically acceptable salt thereof; and
(b) a PD-1 inhibitor.

In some aspects, the cancer is a SMARCA4-deleted cancer.

### BRIEF DESCRIPTION OF THE DRAWINGS

FIG. 1A shows Formula (I)'s effects on activating T cells. Human peripheral blood mononuclear cells (PBMCs) were co-cultured with NCI-H1299 cells (SMARCA4 deleted NSCLC) with Pembrolizumab and/or Formula (I). The cell samples from co-culture were stained for CD3 (T cell marker), CD8 (T cell marker), and CD25 (T cell activation marker) and analyzed by flow cytometry to determine percentages of cells with marker positive populations.

FIG. 1B shows Formula (I)'s effect on modulating cytokine expression in the co-culture system. Supernatant from the co-culture were plated into a 96-well plate and cytokines were analyzed using the MSD U-PLEX Immuno-Oncology Group 1 (Human) kit (MesoScale Discovery, Custom-made kit). Expressions of IFN-gamma, IL-2 and Granzyme B are shown.

FIG. 1C shows the effect of a mouse-active SMARCA2 degrader (Compound 4143) effect on modulating cytokine/chemokine expression levels in murine SMARCA4 KO CT26 colon cancer cells (CRISPR KO clones: A2 and F8). Treated CT26 cells were lysed for mRNA extraction, followed by RT2 profiler PCR array (Immune regulation). Genes significantly changed by Compound 4143 treatment, compared to DMSO control treated cells, were shown.

FIG. 2A shows that Formula (I) promoted T-cell mediated A549 lung cancer cell killing after co-culturing with HLA-matched T cells. Green fluorescence protein (GFP) positive A549 cells were cultured with or without HLA-matched T cells and treated with Formula (I) in the spheroid culture system. Representative figures of the 3D cancer cell spheroids were shown.

FIG. 2B shows quantitated 3D tumor spheroid growth monitored using Incucyte cultured with or without T cells and Formula (I). The data were generated with SMARCA4 deleted A549 cells.

FIG. 2C shows quantitated 3D tumor spheroid growth monitored using Incucyte cultured with human PBMCs. The cells were treated with or without Formula (I) and/or pembrolizumab. The data were generated with SMARCA4 deleted NCI-H1299 and SMARCA4 WT NCI-H1975 cells.

FIG. 3 shows that SMARCA2 degrader Compound 4143 combined with anti-PD-1 mAb treatment improved anti-tumor activity in SMARCA4 deleted CT26 syngeneic tumor model in mice. Balb/c mice were subcutaneously inoculated with SMARCA4 knockout CT26 murine colon cancer cells. When tumor volumes reached 150-200 mm³, mice were randomized and dosed with Compound 4143 and/or anti-PD1 mAb. Tumor volume change from day 10 to day 21 was shown.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub combination.

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

The articles “a,” “an,” and “the” as used herein and in the appended claims are used herein to refer to one or to more than one (e.g., to at least one) of the grammatical object of the article unless the context clearly indicates otherwise. By way of example, “an element” means one element or more than one element.

Where a range of values is provided, it is to be understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

When a range of values is expressed, it is to be understood that the explicitly stated upper and lower limits of the range and all intervening individual values, and all intervening subranges are encompassed by the range. All ranges are inclusive and combinable.

When values are expressed herein as approximations, such as by use of the antecedent “about,” it is understood that the particular value forms another embodiment. In some embodiments, “about” refers to a value with ±10% of the particular value.

The term “administering,” when used in the context of administering a therapeutic agent to a subject, refers to introducing the therapeutic agent into the subject's body. For example, therapeutic agents may be introduced into a subject's body orally, nasally, subcutaneously, intravenously, intravesically, intramuscularly, transdermally, vaginally, rectally or in any combination thereof.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

The term “subject” is used herein to describe an animal, for example, a mammal, to whom treatment with the treatment regimen according to the disclosure is provided. In some aspects, the mammal is a human to whom treatment is provided. In other aspects, the mammal is a non-human to whom treatment is provided.

“Pharmaceutically acceptable” means approved or approvable by a regulatory agency of the United States Federal government or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethane-sulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate, and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols. See for example, Remington, J. P. (2020). Remington, the science and practice of pharmacy, Elsevier Science.

The terms “cancer” and “tumor” are used interchangeably herein and shall be given their ordinary meaning.

As used herein, the term “treatment regimen” refers to coordinated dosages and administration timings of each of Formula (I) (or pharmaceutically acceptable salt thereof) and the PD-1 inhibitor such that Formula (I) (or pharmaceutically acceptable salt thereof) and the PD-1 inhibitor are administered to the subject during an overlapping period of time. Thus, the “treatment regimen” of the disclosure encompasses both concurrent administration of the compound of Formula (I) (or a pharmaceutically acceptable salt thereof) and the PD-1 inhibitor; and sequential administration of the compound of Formula (I) (or a pharmaceutically acceptable salt thereof) and a PD-1 inhibitor. Moreover, the treatment regimen encompasses administration of the compound of Formula (I) (or a pharmaceutically acceptable salt thereof) and a PD-1 inhibitor on the same day; on different days; and any permutation thereof.

As used herein, the term “PD-1 inhibitor” refers to a chemical compound (including biological molecules such as antibodies) that blocks, inhibits, or otherwise interferes with binding of PD-L1 expressed on a cancer cell to PD-1 expressed on an immune cell (T cell. B cell or NKT cell). As such, PD-1 inhibitors include both PD-1 antagonists and PD-L1 antagonists. PD-1 inhibitors useful in the treatment methods of the disclosure include monoclonal antibodies (mAbs) or antigen binding fragments thereof, which specifically binds to PD-1 or PD-L1. Exemplary PD-1 inhibitors include pembrolizumab (Keytruda®), nivolumab (Opdivo®)), cemiplimab (Libtayo®), atezolizumab (Tecentriq®), avelumab (Bavencio®), and durvalumab (Imfinzi®).

As used herein, the term “antibody” shall be given its ordinary meaning and refers to any form of antibody that exhibits the desired biological or binding activity, and includes monoclonal antibodies (including full length monoclonal antibodies), polyclonal antibodies, multispecific antibodies (e.g., bispecific antibodies), humanized antibodies, fully human antibodies, chimeric antibodies, and camelized single domain antibodies.

As used herein, the term “monoclonal antibody” or “mAb” or “Mab”, refers to a population of substantially homogeneous antibodies, i.e., the antibody molecules comprising the population are identical in amino acid sequence except for possible naturally occurring mutations that may be present in minor amounts.

As used herein, unless otherwise indicated, “antibody fragment” or “antigen binding fragment of an antibody” refers to antibody fragments that retain the ability to bind specifically to the antigen to which the full-length antibody binds.

In some aspects, the disclosure is directed to methods of treating cancer in a subject in need thereof, comprising administering to the subject a treatment regimen comprising:
(a) a compound of Formula (I):

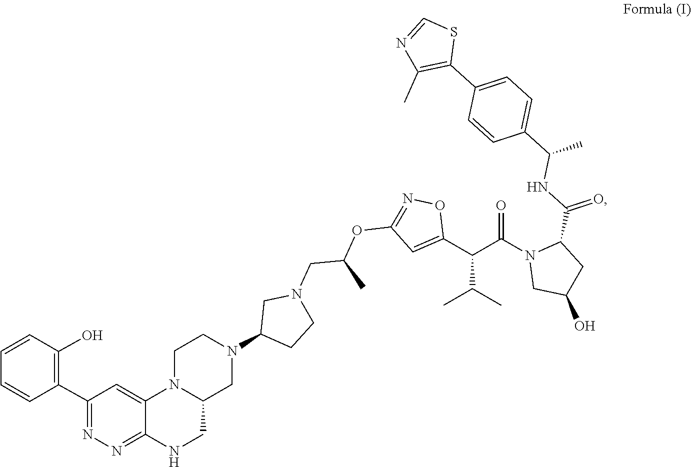 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

or a pharmaceutically acceptable salt thereof; and
(b) a PD-1 inhibitor.

In some embodiments of the disclosed methods, the compound of Formula (I), or a pharmaceutically acceptable salt thereof, and the PD-1 inhibitor are administered concurrently or sequentially.

In some embodiments of the disclosed methods, the compound of Formula (I), or a pharmaceutically acceptable salt thereof, and the PD-1 inhibitor are administered concurrently.

In other embodiments of the disclosed methods, the compound of Formula (I), or a pharmaceutically acceptable salt thereof, and the PD-1 inhibitor are administered sequentially.

In some aspects, the subject is administered the compound of Formula (I), or a pharmaceutically acceptable salt of a compound of Formula (I) (on a Formula (I) basis), in an amount of about 20 mg—about 1000 mg per dose, such as, for example, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg per dose.

In some aspects, the subject is administered the compound of Formula (I), or a pharmaceutically acceptable salt of a compound of Formula (I) (on a Formula (I) basis), in an amount of about 20 mg—about 800 mg per dose.

In some aspects, the compound of Formula (I), or pharmaceutically acceptable salt of a compound of Formula (I), is administered intravenously.

In some embodiments, the compound of Formula (I), or pharmaceutically acceptable salt of a compound of Formula (I), is administered intravenously once per week for 3 weeks.

In some embodiments, the compound of Formula (I), or pharmaceutically acceptable salt of a compound of Formula (I), is administered intravenously in cycles, wherein each cycle comprises administering the compound of Formula (I), or pharmaceutically acceptable salt of a compound of Formula (I), once per week for 3 weeks.

In some aspects of the disclosed methods, the compound of Formula (I), or pharmaceutically acceptable salt of a compound of Formula (I), is administered orally.

In some embodiments, the compound of Formula (I), or pharmaceutically acceptable salt of a compound of Formula (I), is administered orally once per week for 3 weeks.

In some embodiments, the compound of Formula (I), or pharmaceutically acceptable salt of a compound of Formula (I), is administered orally in cycles, wherein each cycle comprises administering the compound of Formula (I), or pharmaceutically acceptable salt of a compound of Formula (I), once per week for 3 weeks.

In some aspects of the disclosed methods, the subject is administered a compound of Formula (I).

In other aspects of the disclosed methods, the subject is administered a pharmaceutically acceptable salt of a compound of Formula (I).

In some aspects of the disclosed methods, the subject is administered a PD-1 inhibitor.

In some embodiments, the PD-1 inhibitor is an antibody.

In some embodiments, the PD-1 inhibitor is a monoclonal antibody, or an antigen binding fragment thereof.

In some embodiments, the PD. 1 inhibitor is pembrolizumab, nivolumab, cemiplimab, atezolizumab, avelumab, or durvalumab.

In some embodiments, the PD-1 inhibitor is pembrolizumab (Keytruda®), nivolumab (Opdivo®), cemiplimab (Libtayo®), atczolizumab (Tecentriq®), avelumab (Bavencio®), or durvalumab (Imfinzi®)).

In some embodiments, the PD-1 inhibitor is pembrolizumab.

In some embodiments, the PD-1 inhibitor is nivolumab.

In some embodiments, the PD-1 inhibitor is cemiplimab.

In some embodiments, the PD-1 inhibitor is atezolizumab.

In some embodiments, the PD-1 inhibitor is avelumab.

In some embodiments, the PD-1 inhibitor is durvalumab.

In some aspects of the disclosed methods, the PD-1 inhibitor is administered intravenously.

In other aspects, the PD-1 inhibitor is administered subcutaneously.

In some aspects of the disclosed methods, each dose of the PD-1 inhibitor is administered in an amount of about 200 mg to about 2000 mg per dose, such as, for example about 200 mg, 210 mg, 220 mg, 230 mg, 240 mg, 250 mg, 260 mg, 270 mg, 280 mg, 290 mg, about 300 mg, 310 mg, 320 mg, 330 mg, 340 mg, 350 mg, 360 mg, 370 mg, 380 mg, 390 mg, about 400 mg, 410 mg, 420 mg, 430 mg, 440 mg, 450 mg, 460 mg, 470 mg, 480 mg, 490 mg, about 500 mg, 510 mg, 520 mg, 530 mg, 540 mg, 550 mg, 560 mg, 570 mg, 580 mg, 590 mg, about 600 mg, 610 mg, 620 mg, 630 mg, 640 mg, 650 mg, 660 mg, 670 mg, 680 mg, 690 mg, about 700 mg, 710 mg, 720 mg, 730 mg, 740 mg, 750 mg, 760 mg, 770 mg, 780 mg, 790 mg, about 800 mg, 810 mg, 820 mg, 830 mg, 840 mg, 850 mg, 860 mg, 870 mg, 880 mg, 890 mg, about 900 mg, 910 mg, 920 mg, 930 mg, 940 mg, 950 mg, 960 mg, 970 mg, 980 mg, 990 mg, about 1000 mg, 1010 mg, 1020 mg, 1030 mg, 1040 mg, 1050 mg, 1060 mg, 1070 mg, 1080 mg, 1090 mg, about 1100 mg, 1110 mg, 1120 mg, 1130 mg, 1140 mg, 1150 mg, 1160 mg, 1170 mg, 1180 mg, 1190 mg, about 1200 mg, 1210 mg, 1220 mg, 1230 mg, 1240 mg, 1250 mg, 1260 mg, 1270 mg, 1280 mg, 1290 mg, about 1300 mg, 1310 mg, 1320 mg, 1330 mg, 1340 mg, 1350 mg, 1360 mg, 1370 mg, 1380 mg, 1390 mg, about 1400 mg, 1410 mg, 1420 mg, 1430 mg, 1440 mg, 1450 mg, 1460 mg, 1470 mg, 1480 mg, 1490 mg, about 1500 mg, 1510 mg, 1520 mg, 1530 mg, 1540 mg, 1550 mg, 1560 mg, 1570 mg, 1580 mg, 1590 mg, about 1600 mg, 1610 mg, 1620 mg, 1630 mg, 1640 mg, 1650 mg, 1660 mg, 1670 mg, 1680 mg, 1690 mg, about 1700 mg, 1710 mg, 1720 mg, 1730 mg, 1740 mg, 1750 mg, 1760 mg, 1770 mg, 1780 mg, 1790 mg, about 1800 mg, 1810 mg, 1820 mg, 1830 mg, 1840 mg, 1850 mg, 1860 mg, 1870 mg, 1880 mg, 1890 mg, about 1900 mg, 1910 mg, 1920 mg, 1930 mg, 1940 mg, 1950 mg, 1960 mg, 1970 mg, 1980 mg, 1990 mg, or about 2000 mg per dose.

In some embodiments, the PD-1 inhibitor is pembrolizumab. Pembrolizumab is marketed in the United States under the trade name

Keytruda® (pembrolizumab).

In some embodiments in which the treatment regimen comprises pembrolizumab, the pembrolizumab is administered in accordance with the March 2024 Revision of the Keytruda® (pembrolizumab) prescribing information, the entirety of which is incorporated by reference herein.

In some embodiments in which the treatment regimen comprises pembrolizumab, the pembrolizumab is intravenously administered to the subject.

In some embodiments in which the treatment regimen comprises pembrolizumab, the pembrolizumab is intravenously administered to the subject in a dose of 200 mg every 3 weeks, or 400 mg every 6 weeks.

In some embodiments in which the treatment regimen comprises pembrolizumab, the pembrolizumab is intravenously administered to the subject in a dose of 200 mg every 3 weeks.

In other embodiments in which the treatment regimen comprises pembrolizumab, the pembrolizumab is intravenously administered to the subject in a dose of 400 mg every 6 weeks.

In some embodiments in which the treatment regimen comprises pembrolizumab, the pembrolizumab is subcutaneously administered to the subject.

In some embodiments in which the treatment regimen comprises pembrolizumab, the pembrolizumab is subcutaneously administered to the subject in a dose of 285 mg every 3 weeks.

In some embodiments, the PD-1 inhibitor is nivolumab. Nivolumab is marketed in the United States under the trade name Opdivo® (nivolumab).

In some embodiments in which the treatment regimen comprises nivolumab, the nivolumab is administered in accordance with the March 2024 Revision of the Opdivo® (nivolumab) prescribing information, the entirety of which is incorporated by reference herein.

In some embodiments in which the treatment regimen comprises nivolumab, the nivolumab is intravenously administered to the subject.

In some embodiments in which the treatment regimen comprises nivolumab, the nivolumab is intravenously administered to the subject in a dose of 240 mg every 2 weeks, or 480 mg every 4 weeks.

In some embodiments in which the treatment regimen comprises nivolumab, the nivolumab is intravenously administered to the subject in a dose of 240 mg every 2 weeks.

In some embodiments in which the treatment regimen comprises nivolumab, the nivolumab is intravenously administered to the subject in a dose of 480 mg every 4 weeks.

In some embodiments in which the treatment regimen comprises nivolumab, the nivolumab is intravenously administered to the subject in a dose of 360 mg every 3 weeks.

In some embodiments in which the treatment regimen comprises nivolumab, the nivolumab is intravenously administered to the subject in a dose of 3 mg/kg every 2 weeks.

In some embodiments in which the treatment regimen comprises nivolumab, the nivolumab is intravenously administered to the subject in a dose of 6 mg/kg every 4 weeks.

In some embodiments in which the treatment regimen comprises nivolumab, the nivolumab is subcutaneously administered to the subject.

In some embodiments, the PD-1 inhibitor is cemiplimab. Cemiplimab is marketed in the United States under the trade name Cemiplimab (Libtayo®).

In some embodiments in which the treatment regimen comprises cemiplimab, the cemiplimab is administered in accordance with the April 2024 Revision of the Cemiplimab (Libtayo®) prescribing information, the entirety of which is incorporated by reference herein.

In some embodiments in which the treatment regimen comprises cemiplimab, the cemiplimab is intravenously administered to the subject.

In some embodiments in which the treatment regimen comprises cemiplimab, the cemiplimab is intravenously administered to the subject in a dose of 350 mg every 3 weeks.

In some embodiments in which the treatment regimen comprises cemiplimab, the cemiplimab is subcutaneously administered to the subject.

In some embodiments in which the treatment regimen comprises cemiplimab, the cemiplimab is subcutaneously administered to the subject in a dose of 350 mg every 3 weeks.

In some embodiments, the PD-1 inhibitor is durvalumab. Durvalumab is marketed in the United States under the trade name Imfinzi® (durvalumab).

In some embodiments in which the treatment regimen comprises durvalumab, the durvalumab is administered in accordance with the April 2024 Revision of the Imfinzi® (durvalumab) prescribing information, the entirety of which is incorporated by reference herein.

In some embodiments in which the treatment regimen comprises durvalumab, the durvalumab is intravenously administered to the subject.

In some embodiments in which the treatment regimen comprises durvalumab, the durvalumab is intravenously administered to the subject in a dose of 10 mg/kg every 2 weeks.

In some embodiments in which the treatment regimen comprises durvalumab, the durvalumab is intravenously administered to the subject in a dose of 1,500 mg every 3 weeks.

In some embodiments in which the treatment regimen comprises durvalumab, the durvalumab is intravenously administered to the subject in a dose of 1,500 mg every 4 weeks.

In some embodiments in which the treatment regimen comprises durvalumab, the durvalumab is intravenously administered to the subject in a dose of 20 mg/kg every 3 weeks.

In some embodiments in which the treatment regimen comprises durvalumab, the durvalumab is intravenously administered to the subject in a dose of 20 mg/kg every 4 weeks.

In other embodiments in which the treatment regimen comprises durvalumab, the durvalumab is subcutaneously administered to the subject.

In some embodiments, the PD-1 inhibitor is atezolizumab. Atezolizumab is marketed in the United States under the trade name Tecentriq® (atezolizumab).

In some embodiments in which the treatment regimen comprises atezolizumab, the atezolizumab is administered in accordance with the April 2024 Revision of the Tecentriq® (atezolizumab) prescribing information, the entirety of which is incorporated by reference herein.

In some embodiments in which the treatment regimen comprises atezolizumab, the atezolizumab is intravenously administered to the subject.

In some embodiments in which the treatment regimen comprises atezolizumab, the atezolizumab is intravenously administered to the subject in a dose of 840 mg every 2 weeks.

In some embodiments in which the treatment regimen comprises atezolizumab, the atezolizumab is intravenously administered to the subject in a dose of 1,200 mg every 3 weeks.

In some embodiments in which the treatment regimen comprises atezolizumab, the atezolizumab is intravenously administered to the subject in a dose of 1,680 mg every 4 weeks.

In other embodiments in which the treatment regimen comprises atezolizumab, the atezolizumab is subcutaneously administered to the subject.

In some embodiments, the PD-1 inhibitor is avelumab. Avelumab is marketed in the United States under the trade name Bavencio® (avelumab).

In some embodiments in which the treatment regimen comprises avelumab, the avelumab is administered in accordance with the March 2024 Revision of the Bavencio® (avelumab) prescribing information, the entirety of which is incorporated by reference herein.

In some embodiments in which the treatment regimen comprises avelumab, the avelumab is intravenously administered to the subject.

In some embodiments in which the treatment regimen comprises avelumab, the avelumab is intravenously administered to the subject in a dose of 800 mg every 2 weeks.

In some embodiments in which the treatment regimen comprises avelumab, the avelumab is subcutaneously administered to the subject.

In some aspects of the disclosed methods, the PD-1 inhibitor is administered by intravenous infusion over a period of about 30 minutes to about 60 minutes.

In some embodiments, the PD-1 inhibitor is administered by intravenous infusion over a period of about 30 minutes.

In some embodiments, the PD-1 inhibitor is administered by intravenous infusion over a period of 30 minutes.

In some embodiments, the PD-1 inhibitor is administered by intravenous infusion over a period of about 60 minutes.

In some embodiments, the PD-1 inhibitor is administered by intravenous infusion over a period of 60 minutes.

In some aspects, the treatment regimen of the disclosure further comprise an additional therapeutic agent. Exemplary additional therapeutic agents include, for example, other anticancer agents (e.g., gemcitabine, doxorubicin, cyclophosphamide, cisplatin, carboplatin, fluorouracil), corticosteroids (e.g., prednisone or dexamethasone), antihistamines (e.g., dexchloropheniramine or diphenylhydramine), H₂ antagonist (e.g., ranitidine), antiemetics, pemetrexed, platinum chemotherapy, carboplatin, paclitaxel or paclitaxel protein-bound, enfortumab vedotin, trastuzumab, fluoropyrimidine-containing chemotherapy, chemoradiotherapy, gemcitabine, cisplatin, lenvatinib, remelimumab-actl, etoposide, carboplatin, cisplatin, bevacizumab, paclitaxel, carboplatin, bevacizumab, cobimetinib, vemurafenib, axitinib, ipilimumab, cabozantinib.

In some aspects, the methods of the disclosure are directed to treating cancer in a subject. In some embodiments, the cancer is one or more of include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present disclosure include, for example, T-lineage acute lymphoblastic leukemia (T-ALL), T-lineage lymphoblastic lymphoma (T-LL), peripheral T-cell lymphoma, adult T-cell leukemia, pre-B ALL, pre-B lymphomas, large B-cell lymphoma, Burkitts lymphoma, B-cell ALL, Philadelphia chromosome positive ALL and Philadelphia chromosome positive CML.

In other embodiments, the cancer is one or more of melanoma, non-small cell lung cancer, head and neck squamous cell cancer, classical hodgkin lymphoma, primary mediastinal large b-cell lymphoma, urothelial cancer, microsatellite instability-high or mismatch repair deficient cancer, microsatellite instability-high or mismatch repair deficient colorectal cancer, gastric cancer, esophageal cancer, cervical cancer, hepatocellular carcinoma, biliary tract cancer, merkel cell carcinoma, renal cell carcinoma, endometrial carcinoma, tumor mutational burden-high cancer, cutaneous squamous cell carcinoma, triple-negative breast cancer, adult classical Hodgkin lymphoma, adult primary mediastinal large b-cell lymphoma, cutaneous squamous cell carcinoma, basal cell carcinoma, non-small cell lung cancer, small cell lung cancer, biliary tract cancers, hepatocellular carcinoma, alveolar soft part sarcoma, metastatic Merkel cell carcinoma, locally advanced or metastatic urothelial carcinoma, advanced renal cell carcinoma, unresectable or metastatic melanoma, adjuvant treatment of melanoma, neoadjuvant treatment of respectable non-small cell lung cancer, metastatic non-small cell lung cancer, malignant pleural mesothelioma, classical hodgkin lymphoma, urothelial carcinoma, microsatellite instability-high or mismatch repair deficient metastatic colorectal cancer, esophageal cancer, gastric cancer, gastroesophageal junction cancer, esophageal adenocarcinoma.

In other embodiments, the cancer is one or more of breast cancer, non-small cell lung cancer, lung squamous cell carcinoma, uterine/endometrio adenocarcinoma, ovarian cancer, lung adenocarcinoma, bladder cancer, skin cancer, hormone refractory prostate cancer, colon adenocarcinoma, gastric adenocarcinoma, pancreatic cancer, AIDS-related Kaposi's sarcoma, or squamous cell carcinoma of the head and neck cancer.

In some embodiments, the cancer is breast cancer or prostate cancer.

In some embodiments, the cancer is breast cancer.

In some embodiments, the cancer is metastatic breast cancer.

In some embodiments, the cancer is prostate cancer.

In some embodiments, the cancer is hormone-refractory metastatic prostate cancer.

In some embodiments, the cancer is non-small cell lung cancer.

In some embodiments, the cancer is adenocarcinoma of the pancreas.

In some embodiments of the disclosed methods, the cancer is a SMARCA4 deficient cancer. As used herein, the term “SMARCA4 deficient” means that the cancer either lacks a SMARCA4 gene, or has a mutated SMARCA4 gene that encodes a fully- or partially-non-functional SMARCA4 protein.

In some embodiments of the disclosed methods, the cancer is not a SMARCA4 deficient cancer.

In some aspects, the methods of the disclosure comprise administering to the subject, in addition to the disclosed treatment regimen, an additional therapeutic agent. Exemplary additional therapeutic agents that may be administered with the disclosed treatment regimen include cyclophosphamide, cisplatin, doxorubicin, prednisone, and fluorouracil.

In some aspects of the methods of the disclosure, administration of the disclosed treatment regimen results in greater tumor growth inhibition than results from administration of either Formula (I) alone or the PD-1 inhibitor alone. As used herein, tumor growth inhibition is calculated as follows: Mean % Inhibition=(mean(C)−mean(T))/mean (C)*100%, wherein C is the tumor volume in the control arm, and T is the tumor volume in the treatment arm.

In some aspects of the methods of the disclosure, administration of the disclosed treatment regimen results in at least 40% tumor growth inhibition, such as, for example, at least 40%, at least 45%, at least 50%, at least 55%, at least 60%, at least 65%, at least 70%, at least 75%, at least 80%, at least 85%, at least 90%, or at least 95% tumor growth inhibition, relative to no treatment.

In some aspects of the methods of the disclosure, the cancer exhibits a complete response (CR) or a partial response (PR) to the administration of the treatment regimen, as evaluated by the Response Evaluation Criteria in Solid Tumors (RECIST) v1.1 criteria.

In some embodiments, the cancer exhibits a complete response (CR) to the administration of the treatment regimen, as evaluated by the Response Evaluation Criteria in Solid Tumors (RECIST) v1.1 criteria.

In other embodiments, the cancer exhibits a partial response (PR) to the administration of the treatment regimen, as evaluated by the Response Evaluation Criteria in Solid Tumors (RECIST) v1.1 criteria.

The disclosure is also directed to the following aspects:

Aspect 1. A method of treating cancer in a subject in need thereof, comprising administering to the subject a therapeutically effective amount of a SMARCA2 inhibitor.

Aspect 2. The method of aspect 1, wherein the SMARCA2 inhibitor comprises Compound I, or a pharmaceutically acceptable salt thereof.

Aspect 3. The method of any one of the preceding aspects, wherein the SMARCA2 inhibitor is administered using a dosing regimen and schedule disclosed herein.

Aspect 4. The method of any one of the preceding aspects, wherein the cancer comprises one or more tumors with loss of SMARCA4.

Aspect 5. The method of any one of the preceding aspects, wherein the cancer comprises one or more tumors with mutated SMARCA4.

Aspect 6. A method of treating cancer in a subject in need thereof, comprising administering to the subject a treatment regimen comprising Compound I, or a pharmaceutically acceptable salt thereof, and a PD-1 or PD-L1 antagonist.

Aspect 7. The method of aspect 6, wherein Compound I, or a pharmaceutically acceptable salt thereof, and the PD-1 or PD-L1 antagonist are administered concurrently or sequentially.

Aspect 8. The method of aspect 6 or aspect 7, wherein the treatment regimen comprises Compound I, or a pharmaceutically acceptable salt thereof, and a PD-1 antagonist.

Aspect 9. The method of aspect 8, wherein the PD-1 antagonist is a monoclonal antibody, or an antigen binding fragment thereof.

Aspect 10. The method of aspect 9, wherein the PD-1 antagonist is an anti-PD-1 antibody.

Aspect 11. The method of any one of aspects 6 to 10, wherein the PD-1 antagonist is pembrolizumab.

Aspect 12. The method of any one of aspect 6 to 11, wherein the cancer comprises one or more tumors with loss of SMARCA4.

Aspect 13. The method of any one of aspect 6 to 11, wherein the cancer comprises one or more tumors with mutated SMARCA4.

### EXAMPLES

### Example 1

### Cell Lines

H1299 (CRL-5803) and H1975 (CRL-5908) were purchased from the American Type Cell Collection. Human Mononuclear Cells (PBMC) (HUMANPBMC-0001989) were purchased from BioIVT. NCI-H1299, NCI-H1975 and all CT26 cells were cultured in RPMI-1640 (ATCC, 30-2001) supplemented with 10% FBS (Gibco, 26140-079) and 1× penicillin-streptomycin (Thermofisher, 15140122). Human Peripheral Bloogd Mononuclear Cells (PBMCs) were cultured in RPMI-1640, 20% FBS, 100U/ml Human Recombinant IL-2 (Gibco, PHC0027) and 1× penicillin-streptomycin. All cells were maintained at 37° C. in 5% CO₂ in a humidified incubator.

### Flow Cytometry

Samples from co-culture were collected in Eppendorf tubes and 500 ul of PBS was added. The samples were centrifuged (300 g, 5 minutes) to wash. Supernatant was removed and the pellet was resuspended in 200 ul FACS buffer. Samples were placed on ice for 15 minutes to block. After blocking, the samples were centrifuged (300 g, 5 minutes). Supernatant was removed and CD25 (BioLegend, 302613), CD8 (BioLegend, 344711), and CD3 (BioLegend, 300329) were added at 1:100 to each sample and incubated on ice for 30 minutes in the dark. 500 ul of FACs buffer was added and the samples were centrifuged (300 g, 5 minutes). Following centrifugation, the supernatant was removed, and the samples were washed again.

Cytokine Analyses using Meso Scale Discovery (MSD)

Supernatant from the co-culture were plated into a 96-well plate and cytokines were analyzed using the MSD U-PLEX Immuno-Oncology Group 1 (Human) kit (MesoScale Discovery, Custom-made kit).

mRNA Expression Analysis by RT2 Profiler PCR Array

SMARCA4 KO A2 and F8 clone cells were treated with Compound 4143. Total RNA was extracted and reverse transcription was performed. The cDNA samples were used for PCR array (Qiagen, RT2 PCR Profiler 330231).

### Results:

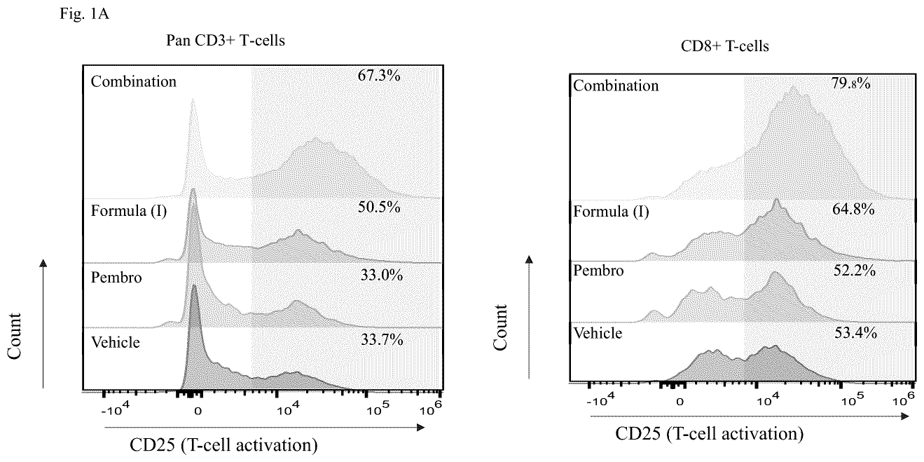

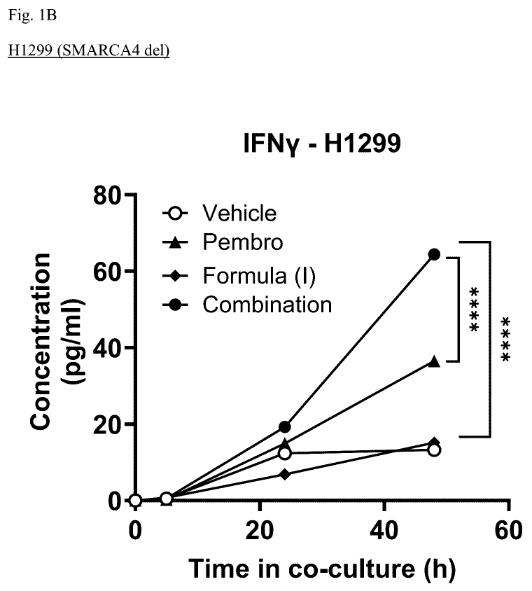

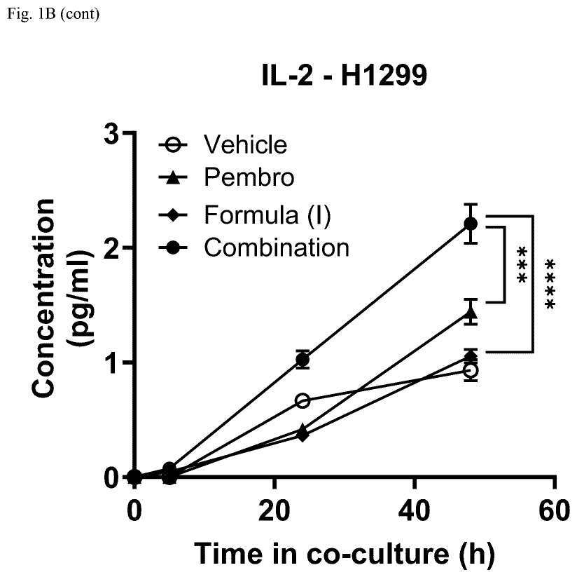

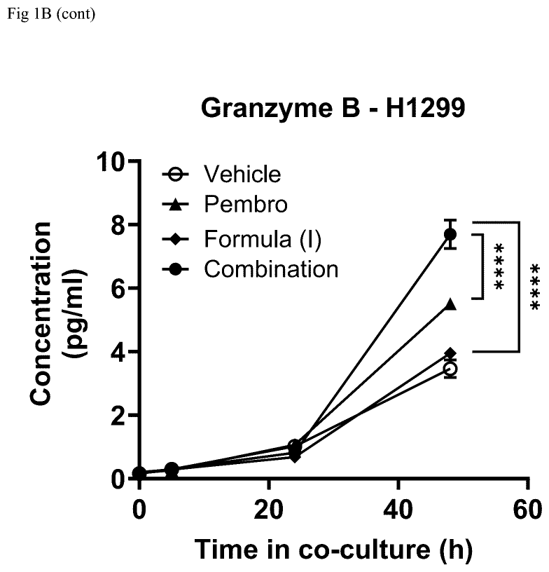

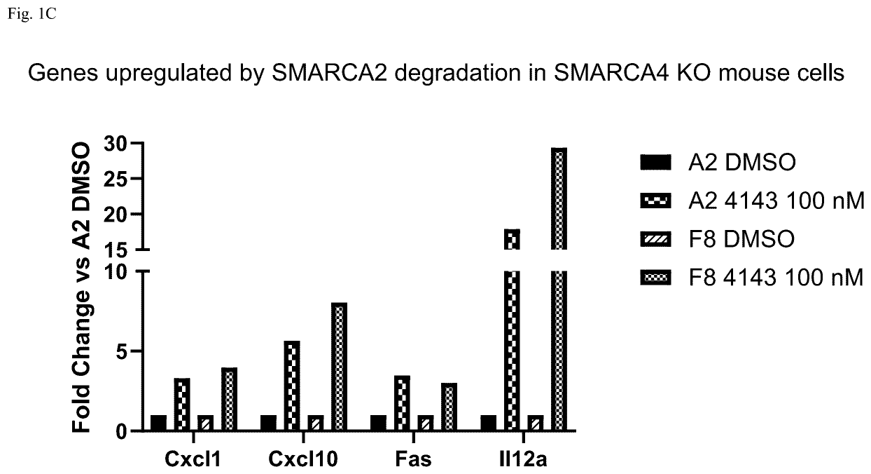

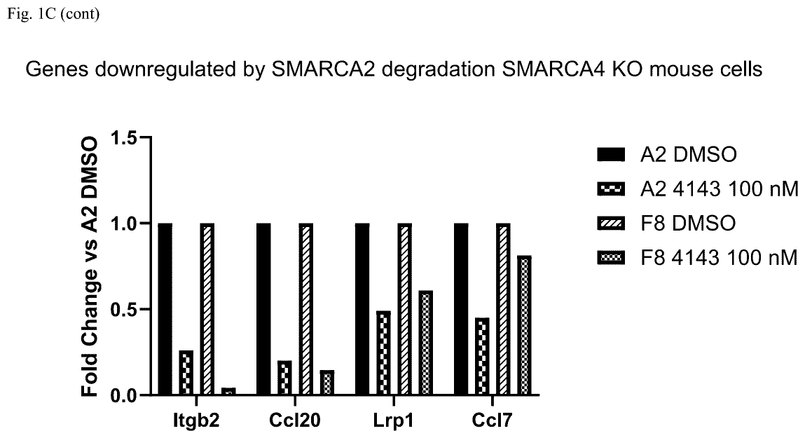

In the co-culture system of SMARCA4 deleted NCI-H1299 cells and human PBMCs, treatment with Formula (I) combined with Pembrolizumab shows increased activated (CD25+) CD3+ T cells and CD8+ T cells (FIG. 1A). Supernatant from the co-cultured cells treated with the combination (conditioned media) contained higher levels of IFN-g, IL-2 and Gramzyme B (FIG. 1B). Degradation of SMARCA2 by Compound 4143 also modulates expression levels of cytokine and chemokine in SMARCA4 KO CT26 murine colon cancer cells. Treatment with Compound 4143 upregulated Cxcl1, Cxcl10, Fas, IL12a and downregulated Itgb2, Cc120, Lrp1 and Ccl7 (FIG. 1C).

### Example 2

### Incucyte Tumor Growth Analyses:

GFP-tagged tumor cells were seeded in 96 well Ultra-Low Adherence (ULA) plates (Corning, 7007) (2000 cells/well) and centrifuged briefly (300 g, 30 seconds) to induce cell aggregation. The cells were incubated at 37° C. overnight to allow complete aggregation into spheroids. Following the addition of immune cells with treatment of Pembrolizumab (MedChemExpress, HY-P9902) (100 ug/ml) and/or Formula (I) (100 nM). Three-dimensional tumor spheroid growth was monitored using Sartorius' Incucyte® SX1 Live-Cell Analysis.

### Incucyte Cytolight Lentivirus for Cytoplasmic Labeling

H1299 and H1975 cells were seeded (500,000 cells/well) in 6 well Tissue Culture Treated plates (Corning 3516) and allowed to aggregate into tumor spheroids for 1-2 hours. Tumor spheroids were treated with 600 ul of Incucyte® Cytolight Lentivirus (Sartorius, 4481) for a MOI of 3 and 8 ug/ml Polybrene Transfection Reagent (Sigma-Aldrich, TR-1003). The cells were incubated at 37° C. overnight. Following incubation, media was removed and replaced with fresh media. GFP expression was monitored using Sartorius' Incucyte® SX1 Live-Cell Analysis System (9600-0031).

### Results:

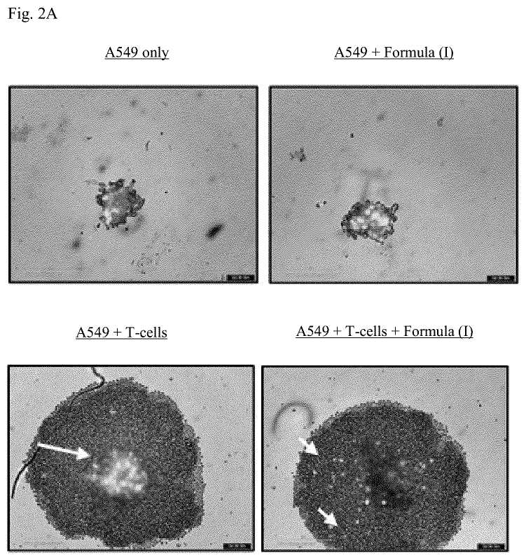

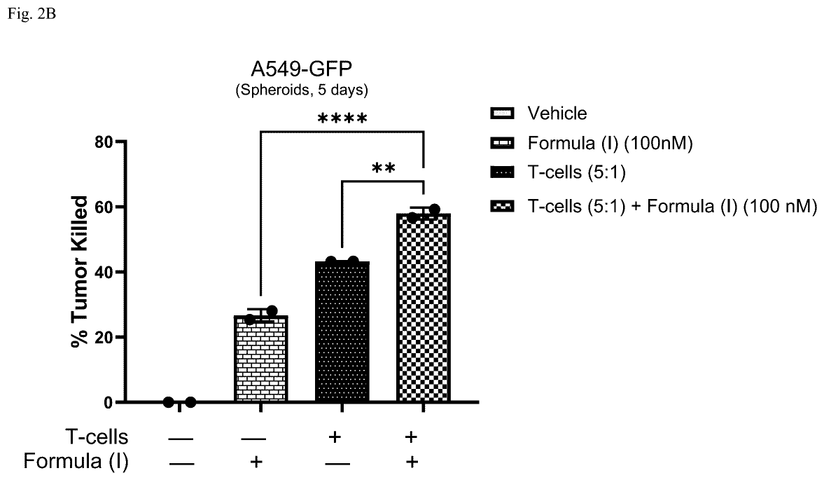

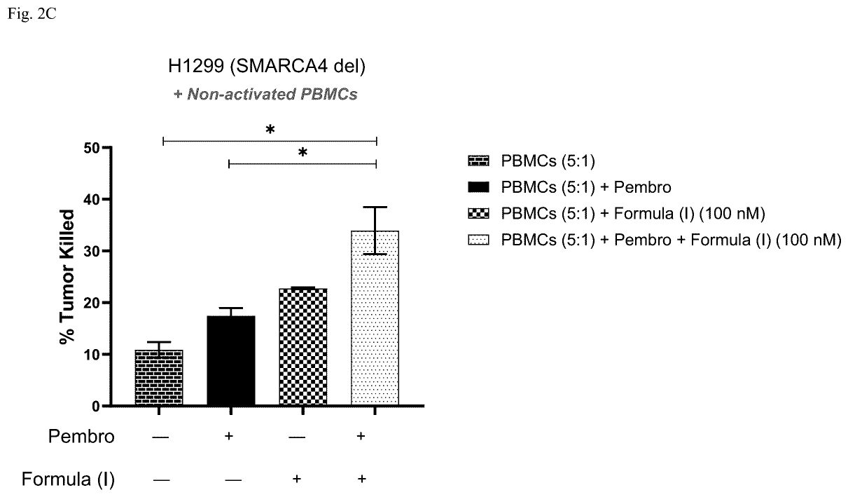

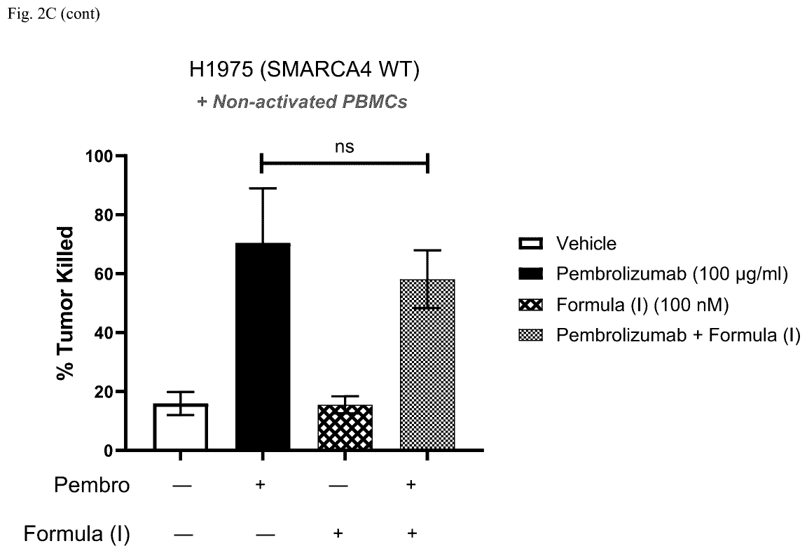

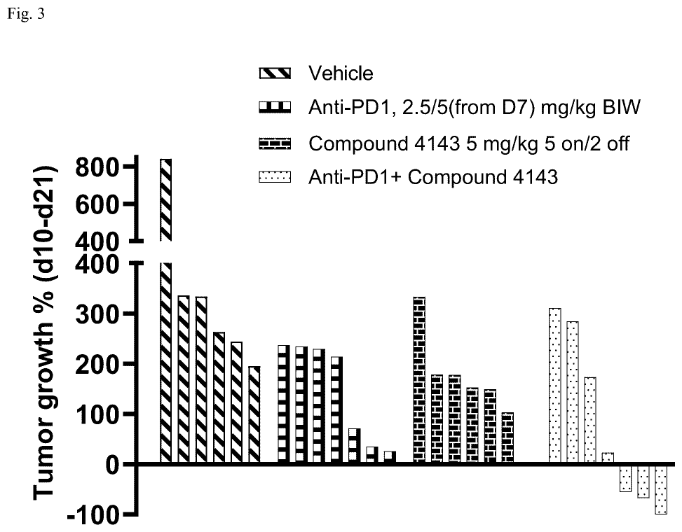

FIG. 2A shows that Formula (I) treatment with HLA-matched T cells inhibited growth of SMARCA4 deleted A549 cancer cells (GFP positive cells) in the 3D spheroid culture system. The quantitated GFP positive A549 cells shows significantly increased A549 killing by Formula (I) treatment in the presence of T cells (FIG. 2B). FIG. 2C shows that combining Formula (I) with pembrolizumab showed more SMARCA4 deleted NCI-H1299 cells cultured with PBMCs, compared to single agent treated groups. In SMARCA4 WT NCI-H1975 cells, the combination effect was not seen.

### Example 3

### Cell Culture:

The CT26 SMARCA4 KO clone A2 cells were maintained in vitro with RPMI1640 medium supplemented with 10% fetal bovine serum at 37° C. in an atmosphere of 5% CO₂ in the air. The cells in exponential growth phase were harvested and quantitated by cell counter before tumor inoculation.

### Tumor Inoculation

Each mouse was inoculated subcutaneously in the right lower flank region with tumor cells (5×10⁵) in 0.1 ml of PBS for tumor development.

### Randomization

The randomization started when the mean tumor size reached approximately 95 mm³. 28 mice were enrolled in each model. All animals were randomly allocated to 4 study groups, 7 mice in each group for each model. Randomization was performed based on “Matched distribution” method (StudyDirector™ software, version 3.1.399.19). The date of randomization was denoted as day 0.

### Test Article Administration

The treatment was initiated on the same day of randomization (day 0) per study design.

### Compound 4143-SMARCA2 Degrader (Active in Mice)

Anti-PD1 (RMP1-14), Leinco, Lot #: 1221L470

### Observation and Data Collection

After tumor cells inoculation, the animals were checked daily for morbidity and mortality. During routine monitoring, the animals were checked for any effects of tumor growth and treatments on behavior such as mobility, food and water consumption, body weight gain/loss (body weights were measured every other day after randomization), eye/hair matting and any other abnormalities. Mortality and observed clinical signs were recorded for individual animals in detail.

Tumor volumes were measured every other day after randomization in two dimensions using a caliper, and the volume was expressed in mm³ using the formula: “V= (L×W×W)/2, where V was tumor volume, L was tumor length (the longest tumor dimension) and W was tumor width (the longest tumor dimension perpendicular to L). Dosing as well as tumor and body weight measurements were conducted in a Laminar Flow Cabinet.

The body weights and tumor volumes were measured by using StudyDirector™ software (version 3.1.399.19).

### Results:

Compound 4143 (mouse active SMARCA2/4 degrader) shows improved antitumor activity when combined with anti-PD1 antibody treatment in a SMARCA4 KO CT26 syngeneic mouse model as measured in tumor volume changes from day 10 and day 21.

1. A method of treating cancer in a subject in need thereof, comprising administering to the subject a treatment regimen comprising:
(a) a compound of Formula (I): 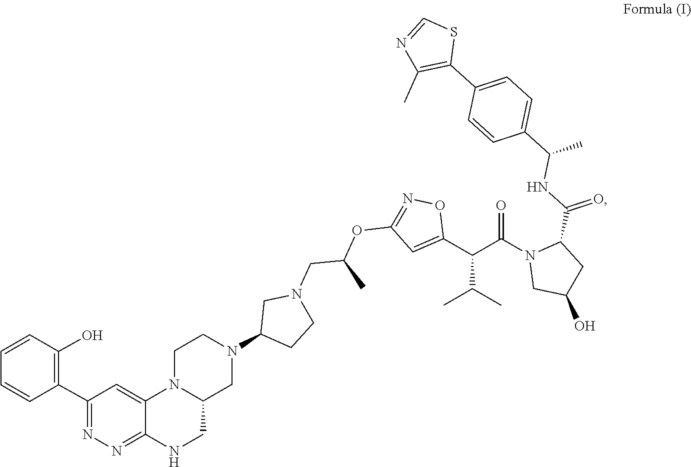 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]
or a pharmaceutically acceptable salt thereof; and
(b) a PD-1 inhibitor.

2. The method of claim 1, wherein the compound of Formula (I), or a pharmaceutically acceptable salt thereof, and the PD-1 inhibitor are administered concurrently or sequentially.

3. The method of claim 1 or claim 2, wherein the PD-1 inhibitor is pembrolizumab, nivolumab, cemiplimab, atezolizumab, avelumab, or durvalumab.

4. The method of claim 3, wherein the PD-1 inhibitor is pembrolizumab.

5. The method of claim 3, wherein the PD-1 inhibitor is nivolumab.

6. The method of claim 3, wherein the PD-1 inhibitor is cemiplimab.

7. The method of claim 3, wherein the PD-1 inhibitor is atezolizumab.

8. The method of claim 3, wherein the PD-1 inhibitor is avelumab.

9. The method of claim 3, wherein the PD-1 inhibitor is durvalumab.

10. The method of claim 1, wherein the subject is administered the compound of Formula (I), or a pharmaceutically acceptable salt of a compound of Formula (I) (on a Formula (I) basis), in an amount of about 20 mg—about 1000 mg per dose.

11. The method of claim 1, wherein the compound of Formula (I), or pharmaceutically acceptable salt of a compound of Formula (I), is administered intravenously.

12. The method of claim 1, wherein the compound of Formula (I), or pharmaceutically acceptable salt of a compound of Formula (I), is administered orally.

13. The method of claim 1, wherein the compound of Formula (I), or pharmaceutically acceptable salt of a compound of Formula (I), is administered once per week for 3 weeks.

14. The method of claim 1, wherein the subject is administered a compound of Formula (I).

15. The method of claim 1, wherein the subject is administered a pharmaceutically acceptable salt of a compound of Formula (I).

16. The method of claim 1, wherein the PD-1 inhibitor is administered intravenously.

17. The method of claim 1, wherein the PD-1 inhibitor is administered in an amount of about 200 mg to about 2000 mg per dose.

18. The method of claim 16, wherein the PD-1 inhibitor is administered by intravenous infusion over about 30 to about 60 minutes.

19. The method of claim 1, wherein the PD-1 inhibitor is administered by intravenous infusion once every 2 weeks, once every 3 weeks, or once every 4 weeks.

20. The method of claim 1, wherein the treatment regimen further comprises an additional therapeutic agent.

21. The method of claim 20, wherein the additional therapeutic agent is gemcitabine, doxorubicin, cyclophosphamide, cisplatin, carboplatin, fluorouracil, prednisone, dexamethasone, dexchloropheniramine, diphenylhydramine, ranitidine, an antiemetic, pemetrexed, paclitaxel, paclitaxel protein-bound, enfortumab vedotin, trastuzumab, fluoropyrimidine-containing chemotherapy, lenvatinib, remelimumab-actl, etoposide, bevacizumab, bevacizumab, cobimetinib, vemurafenib, axitinib, ipilimumab, or cabozantinib.

22. The method of claim 1, wherein the cancer is breast cancer, non-small cell lung cancer, lung squamous cell carcinoma, uterine/endometrio adenocarcinoma, ovarian cancer, lung adenocarcinoma, bladder cancer, skin cancer, hormone refractory prostate cancer, colon adenocarcinoma, gastric adenocarcinoma, pancreatic cancer, AIDS-related Kaposi's sarcoma, or squamous cell carcinoma of the head and neck cancer.

23. The method of claim 1, wherein the cancer is a SMARCA4 deficient cancer.

24. The method of claim 1, wherein administration of the treatment regimen results in greater tumor growth inhibition than results from administration of either Formula (I) alone or the PD-1 inhibitor alone.

25. The method of claim 1, wherein administration of the treatment regimen results in at least 40% tumor growth inhibition relative to no treatment.

26. The method of claim 1, wherein the cancer exhibits a complete response (CR) or a partial response (PR) to the administration of the treatment regimen, as evaluated by the Response Evaluation Criteria in Solid Tumors (RECIST) v1.1 criteria.

27. The method of claim 26, wherein the cancer exhibits a complete response (CR) to the administration of the treatment regimen, as evaluated by the Response Evaluation Criteria in Solid Tumors (RECIST) v1.1 criteria.

28. The method of claim 26, wherein the cancer exhibits a partial response (PR) to the administration of the treatment regimen, as evaluated by the Response Evaluation Criteria in Solid Tumors (RECIST) v1.1 criteria.
